(19) United States
(12) Patent Application Publication
(10) Pub. No.: US 2007/0015839 A1
(43) Pub. Date: Jan. 18, 2007

(54) Daily Dosage Regimen for Treating Diabetes, Obesity, Metabolic Syndrome and Polycystic Ovary Syndrome

(76) Inventors: Franco Folli, Milano (IT); Gilbert R. Gonzales, New York, NY (US); Paolo L. Manfredi, New York, NY (US)

Correspondence Address: WOOD, HERRON & EVANS, LLP, 2700 CAREW TOWER, 441 VINE STREET, CINCINNATI, OH, 45202, US

(21) Appl. No.: 11/428,417

(22) Filed: Jul. 3, 2006

Related U.S. Application Data
(60) Provisional application No. 60/699,182, filed on Jul. 14, 2005.

(51) Int. Cl. A61K 31/155; B65D 85/42

(52) U.S. Cl. 514/635; 206/530

(57) ABSTRACT

A daily dosage regimen for treating diabetes, obesity and metabolic syndrome includes a metabolic pill and one or two biguanide hypoglycemic pills packaged together. The package can include a singe day's regiment, or can include multiple days' regimen. This provides an effective method for treating diabetes, obesity and metabolic syndrome, as well as polycystic ovary syndrome.

### RELATED APPLICATION

This application is a regular utility application of U. S. Provisional Patent Application Ser. No. 60/699,182, filed on Jul. 14, 2005. The entire disclosure of this application is incorporated herein by reference.

### BACKGROUND OF THE INVENTION

Obesity, the Metabolic Syndrome (also referred to as Syndrome X), and Type II Diabetes Mellitus, are very much interrelated. Type II Diabetes Mellitus is caused by a combination of defective insulin secretion and insulin resistance. This results in elevated blood sugar, elevated blood pressure, increased blood lipids and obesity. This determines micro and macro vascular disease with cardiovascular accidents, renal insufficiencies, neuropathy, blindness and higher incidences of infections. Over 120 million people worldwide suffer from diabetes, and 90% of these are Type II Diabetes. Eighty percent of those with diabetes are obese. This has an enormous economic impact in addition to the obvious personal impact on those suffering from this disease.

The Metabolic Syndrome is characterized by individuals having abdominal obesity, high triglycerides, low HDL cholesterol, high blood pressure and high fasting glucose levels. A diagnosis of Metabolic Syndrome is made when three or more of these factors are established. It is believed that as many as 25% of the general population in the United States suffer from Metabolic Syndrome. In addition to being a direct cause of death, those suffering from Metabolic Syndrome have a significantly increased risk of developing Type II Diabetes with all of its complications, as well as other serious cardiovascular complications.

Finally, in the United States, over 61 % of the population is obese or overweight. Fifty-eight million people are overweight, forty million are characterized as obese, and three million are considered morbidly obese.

A somewhat related malady is polycystic ovary syndrome. Polycystic ovary syndrome is characterized by anovulation (irregular or absent menstrual periods) and hyperandrogenism (elevated serum testosterone and androstenedione). Patients with this syndrome may complain of abnormal bleeding, infertility, obesity, excess hair growth, hair loss and acne. Insulin resistance, high blood pressure, high serum lipids, increased risk for cardiovascular disease and increased risk of developing Type II Diabetes are also important features.

Polycystic ovary syndrome is estimated to affect about half as many or approximately 10% of women.

One of the major biochemical features of polycystic ovary syndrome is insulin resistance accompanied by compensatory hyperinsulinemia (elevated fasting blood insulin levels). Hyperinsulinemia has also been associated with high blood pressure and increased clot formation and appears to be a major risk factor for the development of heart disease, stroke and Type II Diabetes.

The medical literature suggests that the endocrinopathy in most patients with polycystic ovary syndrome can be resolved with insulin lowering therapy.

Those people suffering from diabetes generally are treated with a number of different agents, in particular hypoglycemic agents, blood pressure medication, as well as cholesterol-lowering drugs. It is common to use multiple different drugs to treat Type II Diabetes. Several drug combinations have been produced.

Such drug combinations are disclosed in Ikeda U.S. Pat. No. 5,952,356, Adjei U.S. Pat. No. 6,524,621, Chaudhari U.S. pending application 2003/0219482 A1, Jaen et al U.S. application 2002/0037928 A1; Fine et al U.S. Pat. No. 6,376,594, Pierson U.S. Pat. No. 6,693,094, Whitcomb U.S. Pat. No. 6,011,049, Saho et al U.S. Pat. No. 6,645,997; Chungi U.S. Pat. No. 6,669,955; and Luskey U.S. Pat. No. 6,646,004. Further, Liang et al U.S. Pat. No. 6,576,256 discloses a combination of a cholesterol-lowering agent, a renin-angiotensin system inhibitor and aspirin. Sethi et al U.S. Pat. No. 6,489,345 discloses a combination of E vitamins and hypoglycemic drug, and Doebbner et al U.S. Pat. No. 5,847,008 discloses a variety of combinations with acetyl phenols. Other pending applications include 2003/0149111; 2003/0114469; 2003/0149111; and 2003/0158090.

Many of these combinations do not include a hypoglycemic agent. Further, others will include hypoglycemic agents which are unsuitable for treatment of Metabolic Syndrome or obesity. The biguanides are used to treat diabetes, and can also be useful in treating obesity and Syndrome X. However, other hypoglycemic agent such as the sulfonvlureas the alpha glucosidase inhibitors, the glitazones, and the meglitinides will actually induce hypoglycemia in patients who are not suffering from Type II Diabetes. Biguanides have been combined with these other hypoglycemic agents for treatment of Type II Diabetes. Other combination therapies fail to adequately address all of the comorbidities associated with Type II Diabetes with one medicine. Compliance is a critical problem for proper treatment of diabetes. Only a fraction of patients with diabetes is compliant with prescribed regiments. Individuals required to take five or six different pills, either at once or at different times of the day, are less likely to do so. Thus, reducing the number of pills, or, in other words, combining drugs into a single dosage will greatly improve compliance.

Further, the biguanides, metformin in particular, are administered in large dosages. Therefore, administering 1 day's dosage in combination with other drugs in a pill may be impractical.

### SUMMARY OF THE INVENTION

The present invention is premised on the realization that a combination therapy, that is, a metabolic pill, effective for treatment of diabetes, the Metabolic Syndrome and obesity as well as their complications, is provided by utilizing a combination of a biguanide hypoglycemic agent with a lipid lowering agent, a blood pressure lowering agent, optionally an anti-platelet agent, and optionally a combination of vitamins and supplements which have been shown to prevent atherosclerosis and infections. This metabolic pill is packaged in a single daily dose package such as a blister pack together with additional biguanide hypoglycemic agent to provide a daily drug regimen. A single package can contain a 1 day's prescribed drugs, or, the package can contain drugs for a plurality of days, such as a week or a month,separated on a daily basis.

Preferably, the metabolic pill used in the present invention includes metformin as the hypoglycemic agent, simvastatin as the cholesterol lowering agent, and a renin-angiotensin system inhibitor such as lisinopril as the blood pressure lowering agent. The preferred anti-platelet agent is aspirin. These may be combined with one or more of the following: a folate, vitamin B6, B12 and other supplements such as asparginin, beta-carotene, vitamins A, C, D, E, K, and polyunsaturated fatty acids.

The objects and advantages of the present invention will be further appreciated in light of the following detailed description and drawings.

### BRIEF DESCRIPTION OF THE DRAWINGS

FIG. 1 is a diagrammatic view of a blister package incorporating the present invention; and

FIG. 2 is an alternate embodiment of the present invention.

### DETAILED DESCRIPTION OF THE INVENTION

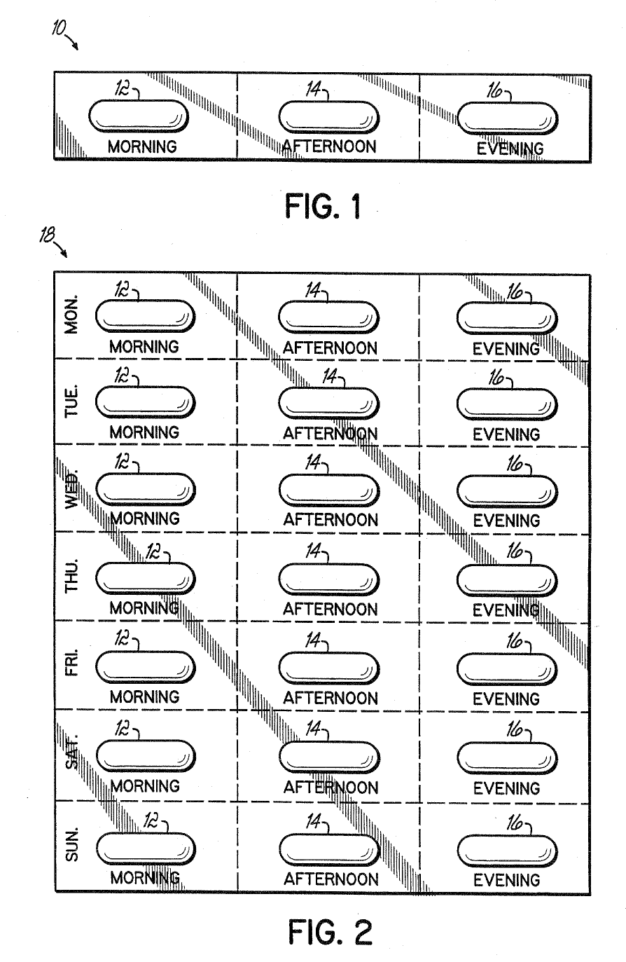

As shown in FIG. 1, the present invention is a daily drug regimen in a single package 10. The package includes two doses of hypoglycemic agent 12,14 (metformin) to be taken at two different times, and a metabolic pill 16 to be taken at a third time. As shown in FIG. 2, a package 18 can be prepared with a week's supply of drugs separated for daily administration, as in FIG. 1. It should be noted that a package containing two daily dosages per day of the hypoglycemic agent are shown. However, with many patients, only one dosage of the hypoglycemic agent may be required in addition to the metabolic pill. Also, pills 12 and 14 may contain different amounts of the active component.

The metabolic pill is a single pill or capsule, which includes the biguanide hypoglycemic agent in combination with additional pharmaceuticals including a cholesterol lowering agent, a blood pressure lowering agent and, optionally, an anti-platelet agent, vitamins and supplements. This composition can be used whenever clinically appropriate to treat Type II Diabetes and, when appropriate, the Metabolic Syndrome and obesity.

The primary component of the metabolic pill is a hypoglycemic drug and, in particular, a biguanide. Metformin is the preferred oral hypoglycemic agent. This is also the active component of the first two pills 12,14 in package 10, i.e., the daily unit package.

A variety of different blood pressure lowering agents can be used in the metabolic pill. These include the renin angiotensin system inhibitors, beta-blockers such as atenolol, diuretics such as hydrochlorothiazide, and calcium channel antagonists, for example, nifedipine.

The renin angiotensin system inhibitors include ACE inhibitors which inhibit the conversion of angiotensin I to angiotensin II, angiotensin II receptor antagonists and renin inhibitors. The ACE inhibitors are preferred inhibitors of the renin angiotensin systems for use in the present invention. The ACE inhibitors include sulfhydryl containing ACE inhibitors including captopril and agents that are structurally related to captopril such as fentialpril, pivalopril, zofenopril, and alacepril. Other ACE inhibitors include the dicarboxyl-containing ACE inhibitors including amalopril, lisinopril, benazapril, quinapril, moexipril, ramipril, spirapril, perindopril, indolapril, pentopril, and cilazapril. Phosphorus-containing ACE inhibitors can also be used, such as fosinopril and ACE inhibitors structurally related thereto.

The preferred ACE inhibitors are captopril, silizopril, delapril, analopril, fentiapril, fosinopril, endolapril, lisinopril, perindopril, pivalopril, quinapril, ramipril, spirapirl, trandolapril and zofinopril. Particularly preferred are captopril, enalipril, fosinopril, lisinopril, quinapril, ramipril, and trandolapril. Most preferred are lisinopril and ramipril.

Examples of ACE/NEP inhibitors for use herein include, without limitation, those disclosed in U.S. Pat. Nos. 5,508,272, 5,362,727, 5,366,973, 5,225,401, 4,722,810, 5,223,516, 5,552,397, 4,749,688, 5,504,080, 5,612,359 and 5,525,723, the disclosures of which are hereby incorporated by reference in their entirety. Preferred are those ACE/NEP inhibitors that are designated as preferred in the above U.S. patents. Particularly preferred are the ACE/NEP inhibitors omapatrilat and MDL100240, disclosed in U.S. Pat. No. 5,430,145.

A second type of renin-angiotensin system inhibitors are the angiotensin II receptor antagonists. Examples of angiotensin II receptor antagonists suitable for use herein are saralasin (including saralasin acetate), candesartan (including candesartan cilexetil), CGP-63170, EMD-66397, KT3-671, LRB/081, valsartan, A-81282, BIBR-363, BIBS-222, BMS-184698, CV1194, EXP-3174, KW-3433, L-161177, L-162154, LR-B/057, LY-235656, PD150304, U-96849, U-97018, UP-275-22, WAY-126227, WK-1492.2K, YM-31472, losartan (including losartan potassium), E-4177, EMD-73495, eprosartan, HN-65021, irbesartan, L-159282, ME-3221, SL-91.0102, tasosartan, telmisartan, UP-269-6, YM-358, CGP-49870, GA-0056, L-159689, L-162234, L-162441, L-163007, PD-123177, A81988, BMS-180560, CGP-38560A, CGP-48369, DA-2079, DE-3489, DuP-167, EXP-063, EXP-6155, EXP-6803, EXP-7711, EXP-9270, FK-739, HR-720, ICI-D6888, ICI-D7155, ICI-D8731, isoteoline, KRI-1177, L-158809, L-158978, L-159874, LR B087, LY-285434, LY-302289, LY-315995, RG-13647, RWJ-38970, RWJ-46458, S-8307, S-8308, saprisartan, sarmesin, WK-1360, X-6803, ZD-6888, ZD-7155, ZD-8731, BIBS39, CI-996, DMP-81 1, DuP-532, EXP-929, L163017, LY-301875, XH-148, XR-510, zolasartan, and PD-123319. These are disclosed in U.S. Pat. No. 6,576,256, the disclosure of which is also incorporated herein by reference.

Preferred angiotensin II receptor antagonists include losartan (which is the prototype and best known angiotensin II receptor antagonist), irbesartan, eprosartan, candesartan, valsartan, telmisartan, zolasartan, and tasosartan. Particularly preferred is losartan.

A third type of angiotensin system inhibitor are the renin inhibitors, these are compounds that inhibit renin activity and include renin antibodies, analogues of prosegment of renin, analogs of pepstatin, and analogs of the renin substrate angiotensinogin. As most of these compounds are peptides, they tend to have low oral bioavailability. Non-peptide renin inhibitors are of the most interest. Preferred inhibitors are remikiren, A-72517, and A-74273, with remikiren being preferred.

The preferred dosage for each of these components obviously will depend on the particular pharmaceutical. The effective dosage ranges for these compounds are well known.

The third component of the metabolic pill is a blood lipid lowering agent. There are many different blood lipid lowering agents. These include, HMG CoA reductase inhibitors, bile acid sequestrants, probucol, and fibric acid agents. The HMG CoA reductase inhibitors include atorvastatin, cerivistatin, fluindostatin, fluvastatin, lovastatin, mevastatin, pravastatin, simvastatin, and velostatin. The preferred HMG CoA reductase inhibitors are simvastatin pravastatin, lovastatin, and atorvastatin. The bile acid sequestrants include cholestyramine, colestipol, and colesevelam. The fibric acid agents include clofibrate, fenofibrate, and gemfibrozil.

Again, the dosage rate for each of these components will be determined by the particular lipid lowering agent selected. Generally, the minimum dose will be designed for individuals with normal to slightly raised lipid levels.

A further optional component of the metabolic pill is an anti-platelet drug. The preferred anti-platelet drug is aspirin. Other salicylates can be used such as magnesium salicylate. Further, there are other anti-platelet aggregating agents, such as anangrelide, dipyridamole, clopidogrel, and ticlopidine. Further, cyclooxygenase inhibitors can be used, including nonstearoidal anti-inflammatory drugs such as ibuprofen, sulindac, sulindac sulfide, sulindac sulfone, flurbiprofen, indomethacin, naproxen, meclafenamic acid, and piroxicam. These should be provided in a dosage effective to inhibit platelet degradation.

The metabolic pill may include various vitamins and supplements shown to prevent atherosclerosis and to prevent infections. One preferred vitamin is folic acid, a folate, or folinic acid, commonly referred to as a folate. Suitable folates include 5-methyl tetrahydrofolic acid, tetrahydrofolic acid, and 5-formyl tetrahydrofolic acid.

Vitamin B components should be included such as vitamin B6 (pyridoxine), and vitamin B12. Other vitamins and minerals include beta-carotine and other carotinoids, vitamin A, vitamin C, vitamin D, vitamin E, vitamin K, zinc, polyunsaturated fatty acids, arginin, as well as its isomers.

A preferred general formulation for the metabolic pill would include:

| | |
|---|---|
| metformin | 125-2000 mg, preferably 500 mg |
| simvastatin | 2.5-160 mg, preferably 20 mg |
| lisinopril | 1.38-60 mg, preferably 40 mg |
| aspirin | 0-1000 mg |

In the daily package, the first two daily pills 12 and 14 will be the hypoglycemic agent. The hypoglycemic agent will preferably be metformin at a dosage of 125-2000 mg, preferably 500 mg.

The blister packs shown in FIGS. 1 and 2 show designations for morning, afternoon and evening pills. This can also be found in braille on the blister pack along side the pills. The morning 12 and afternoon 14 pills for each of the days, for example, could be the hypoglycemic agent or metformin and in a desired dose such as 500 mg. The metabolic pill 16, which would be administered in the evening dose, would be a combination of the biguanide hypoglycemic agent, the blood pressure lowering agent, such as lisinopril, and the lipid lowering agent, such as simvastatin.

Lisinopril may be administered in any dosage from 2.5 mg up to 60 mg, and the simvastatin may be administered in dosages from 2.5 mg to 160 mg. The metformin may be administered from 125 mg to 2000 mg. Therefore, any combination of these different components can be used for formulate the metabolic pill. Prefereably, the metabolic pill will also include aspirin in a range of from 10 mg to 1000 mg, preferably about 81 mg.

Thus, with the present invention, the patient would have an entire day's pills in a blister pack, as shown in FIG. 1. In the morning, the patient would take the first pill, which would be 500 mg of metformin. At lunch he/she would take the second pill, which would also be 500 mg of metformin. In the evening he/she would take the metabolic pill, which would include metformin, lisinopril and simvastatin. The embodiment shown in FIG. 2 would segregate the regimen by day.

The combination of the metformin, lisinopril and simvastatin lipid lowering agent is particularly effective in treating diabetes, syndrome X, and obesity. The blister packaging, or daily dosage packaging allows a significantly larger dosage of metformin. This combination of drugs in a single daily dosage package greatly improve convenience for the patent and thus should also improve compliance, which is absolutely critical for treating any of these maladies.

This has been a description of the present invention along with the preferred method of practicing the present invention. However, the invention itself should only be defined by the appended claims,

Wherein we claim:

1. A package containing a single day's dosage of medicine,: said package including an initial pill to be administered in the morning, and a final pill to be taken in the evening, the initial pill containing a biguanide hypoglycemic agent and the final pill containing a metabolic pill, said metabolic pill comprising a combination of a biguanide hypoglycemic agent, a lipid lowering agent and a blood pressure lowering agent.

2. The package claimed in claim 1 further comprising an intermediate pill comprising said biguanide hypoglycemic agent.

3. The product claimed in claim 1 wherein said packaging is a blister package.

4. The package claimed in claim 3 wherein said blister package comprises multiple days dosages, each dosage in a separate row on said packaging.

5. The package of claim 1 wherein said lipid lowering agent is simvastatin.

6. The package of claim 5 wherein said blood pressure lowering agent is lisinpril.

7. The package of claim 1 wherein said metabolic pill further comprises aspirin.

8. A method of treating one of metabolic syndrome, syndrome X, obesity, type II diabetes, or polycystic ovary syndrome comprising ingesting a first pill in the morning and a second pill in the evening said first pill comprising a biguanide hypoglycemic agent;
a second pill comprising a metabolic pill said metabolic pill including a biguanide hypoglycemic agent, a lipid lowering agent, and a blood pressure lowering agent.

9. The method claimed in claim 8 wherein said method further comprises ingesting a third pill after said first pill and before said second pill wherein said third pill comprises a biguanide hypoglycemic agent.

10. The method claimed in claim 8 wherein said lipid lowering agent is simvastatin.

11. The method claimed in claim 8 wherein said blood pressure lowering agent comprises lisinopril.

12. The method claimed in claim 8 wherein said metabolic pill further includes aspirin.
